(19) United States
(12) Patent Application Publication
(10) Pub. No.: US 2001/0036924 A1
(43) Pub. Date: Nov. 1, 2001

(54) Chemical complex comprising a substituted pyridine carboxy derivative and a glucosaminoglycan

(76) Inventor: Morten Sloth Weidner, Virum (DK)

Correspondence Address: BIRCH STEWART KOLASCH & BIRCH, PO BOX 747, FALLS CHURCH, VA, 22040-0747, US

(21) Appl. No.: 09/813,723

(22) Filed: Mar. 21, 2001

Related U.S. Application Data
(60) Provisional application No. 60/191,689, filed on Mar. 23, 2000.

(30) Foreign Application Priority Data
Mar. 21, 2000 (DK) PA2000 00467

(51) Int. Cl. C07H 5/04; C07H 5/06; C08B 37/00; A61K 31/7008

(52) U.S. Cl. 514/42; 514/54; 514/62; 536/18.7; 536/55.2

(57) ABSTRACT

The present invention relates to a chemical composition comprising an optionally substituted pyridine carboxy derivative and a glucosaminoglycan and a pharmaceutical composition or a dietary supplement comprising an optionally substituted pyridine carboxy derivative and a glucosaminoglycan and to the use of such compositions for the preparation of a medicament or a dietary supplement for immunomodulation in a mammal and the suppression of hypersensitivity and/or inflammatory reaction.

### FIELD OF THE INVENTION

The present invention relates to a chemical composition comprising an optionally substituted pyridine carboxy derivative and a glucosaminoglycan and a pharmaceutical composition or a dietary supplement comprising an optionally substituted pyridine carboxy derivative and a glucosaminoglycan and to the use of such compositions for the preparation of a medicament or a dietary supplement for immunomodulation in a mammal and the suppression of hypersensitivity and/or inflammatory reaction.

### BACKGROUND OF THE INVENTION

Hypersensitivity is defined as a state of altered reactivity in which the body reacts with an exaggerated immune response to a substance (antigen). Hypersensitivity may be caused by exogenous or endogenous antigens.

Hypersensitivity reactions underlie a large number of diseases. Among these, allergic and autoimmune conditions are of great importance. A classification of hypersensitivity diseases is given in the textbook Clinical Medicine (Kumar, P. and Clark, M.: “Clinical Medicine”, 3rd edition, p. 147-150, 1994, Bailliere Tindall, London).

Type I hypersensitivity reactions (IgE mediated allergic reactions) are caused by allergens (specific exogenous antigens), e.g. pollen, house dust, animal dandruff, moulds, etc. Allergic diseases in which type I reactions play a significant role include asthma, eczema (atopic dermatitis), urticaria, allergic rhinitis and anaphylaxis.

Type II hypersensitivity reactions are caused by cell surface or tissue bound antibodies (IgG and IgM) and play a significant role in the pathogenesis of myasthenia gravis, Goodpasture's syndrome and Addisonian pernicious anaemia.

Type III hypersensitivity reactions (immune complex) are caused by autoantigens or exogenous antigens, such as certain bacteria, fungi and parasites. Diseases in which type III hypersensitivity reactions play a significant role include lupus erythematosus, rheumatoid arthritis and glomerulonephritis.

Type IV hypersensitivity reactions (delayed) are caused by cell or tissue bound antigens. This type of hypersensitivity plays a significant role in a number of conditions, e.g. graft-versus-host disease, leprosy, contact dermatitis and reactions due to insect bites.

A number of drug classes are available for the treatment of hypersensitivity reactions. Among these the corticosteroids are some of the most widely used drugs. Corticosteroids primarily exert their pharmacological action by non-selectively inhibiting the function and proliferation of different classes of immune cells resulting in suppression of hypersensitivity reactions. Unfortunately, the corticosteroids are associated with a number of serious side effects, e.g. immuno-suppression, osteoporosis and skin atrophy.

Glucosaminoglycans, formerly known as mucopolysaccharides, are components of various tissues in numerous animals, both vertebrates and invertebrates. Important examples are the Chondroitin sulfates and keratan sulfates of connective tissue, the dermatan sulfates of skin, and hyaluronic acid. Thus chondroitin sulfate is a major component of articular cartilage. Chondroitin sulfate containing glycoproteins act as the flexible matrix between protein filaments in cartilage. Administration of these substances in pharmacological doses has been found to possess some therapeutic potential in osteoarthritis and therefore chondroitin sulfate is used as a chondroprotective agent.

### SUMMARY OF THE INVENTION

It has been found by the present inventor that a chemical complex, a pharmaceutical composition, a dietary supplement or a cosmetic comprising an optionally substituted pyridine carboxy derivativeand a glucosaminoglycan or a fragment or derivative thereof and optionally a pharmaceutically acceptable carrier significantly suppresses hypersensitivity reactions.

Compared to existing therapeutic agents, such as corticosteroids or non-steroidal anti-inflammatory drugs, the chemical complexes, pharmaceutical compositions and dietary supplements according to the present invention have the advantage of not being likely to be associated with any serious side effects, as all of their components are non-toxic and well tolerated by the organism in the pharmacologically relevant doses.

Due to the pharmacological effects mentioned above, the chemical complexes, pharmaceutical compositions and dietary supplements according to the invention can be employed for the following therapeutic applications:

Immunomodulation;

Treatment or prevention of hypersensitivity diseases;

Treatment or prevention of IgE mediated allergic reactions and conditions;

Treatment or prevention of autoimmune disorders;

Chondroprotection;

Alleviation of pain.

Accordingly, the present invention provides a pharmaceutical composition or a dietary supplement comprising: A chemical complex essentially comprising:

i) an optionally substituted pyridine carboxy derivative according to formula 1

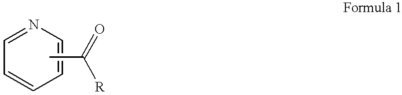

wherein R may be selected from OH; OR′; NH₂; NHR′; NR′R″, O⁻Y⁺, and halogen, wherein R′ and R″ may independently be selected from optionally substituted C₁-C₂₀ alkyl; and Y is a base addition salt of the free carboxylate; and

ii) a glucosaminoglycan or a fragment or derivative thereof.

Furthermore, the present invention provides the use of a chemical complex or a composition comprising an optionally substituted pyridine carboxy derivative and a glucosaminoglycan as described above and optionally a pharmaceutically acceptable carrier for the preparation of a medicament for immunomodulation in a mammal, for the suppression of hypersensitivity reactions in a mammal, such as IgE mediated allergic reactions, and autoimmune reactions in a mammal, and for the alleviation of pain in a mammal, the mammal preferentially being a human.

Thus, according to the invention a chemical complex or a composition comprising an optionally substituted pyridine carboxy derivative and a glucosaminoglycan as described above and optionally a pharmaceutically acceptable carrier can be used in a method for the treatment or prevention of a hypersensitivity disease in a mammal, said method comprising administering said chemical complex or said composition to said mammal; and the invention comprises the use of said chemical complex or said composition for the preparation of a medicament for the treatment or prevention of hypersensitivity diseases in a mammal.

Also, according to the invention a chemical complex or a composition comprising an optionally substituted pyridine carboxy derivative and a glucosaminoglycan as described above and a pharmaceutically acceptable carrier can be used in a method for the treatment or prevention of an autoimmune disorder in a mammal, said method comprising administering said chemical complex or said composition to said mammal; and the invention comprises the use of said chemical complex or said composition for the preparation of a medicament for the treatment or prevention of autoimmune disorders in a mammal.

Further, according to the invention a chemical complex or a composition comprising an optionally substituted pyridine carboxy derivative and a glucosaminoglycan as described above and optionally a pharmaceutically acceptable carrier can be used in a method for the treatment or prevention of an IgE mediated allergic reaction or condition in a mammal, said method comprising administering said chemical complex or said composition to said mammal; and the invention comprises the use of said chemical complex or said composition for the preparation of a medicament for the treatment or prevention of IgE mediated allergic reactions and conditions in a mammal.

Also, according to the invention a chemical complex or a composition comprising an optionally substituted pyridine carboxy derivative and a glucosaminoglycan as described above and optionally a pharmaceutically acceptable carrier can be used in a method for the alleviation of pain in a mammal, said method comprising administering said chemical complex or said composition to said mammal; and the invention comprises the use of said chemical complex or said composition for the preparation of a medicament for the alleviation of pain in a mammal.

### DETAILED DESCRIPTION OF THE INVENTION

It has been found by the present inventor that a chemical complex or a composition comprising an optionally substituted pyridine carboxy derivative and a glucosaminoglycan a fragment thereof or a derivative thereof and optionally a pharmaceutically acceptable carrier significantly suppresses hypersensitivity reactions.

Such chemical complexes or compositions are novel and provide a surprisingly good anti-hypersensitivity, anti-inflammatory and chondroprotective effect with a surprisingly good safety profile. Thus the chemical complexes or compositions of the invention are virtually non-toxic and yet very therapeutically effective. The present inventor puts forward the hypothesis that the very beneficial therapeutic index of the compositions of the invention compared to single chemical anti-hypersensitivity drugs is due to the more complex nature of the compositions of the invention, giving a lower toxic load on the body of any single chemical compound and yet giving a surprisingly good therapeutic effect, due to synergistic effects between the components of the compositions.

More specifically, the above mentioned chemical complexes or compositions of the invention provide the following pharmacological effects upon administration to the living organism:

Immunomodulation;

Treatment or prevention of hypersensitivity diseases;

Treatment or prevention of IgE mediated allergic reactions and conditions;

Treatment or prevention of autoimmune disorders;

Chondroprotection;

Alleviation of pain.

Accordingly, the present invention provides a pharmaceutical composition or a dietary supplement comprising: A chemical complex essentially comprising:

i) an optionally substituted pyridine carboxy derivative according to formula 1

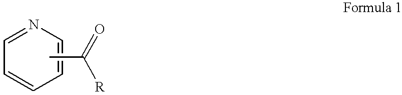

wherein R may be selected from OH; OR′; NH₂; NHR′; NR′R″, O⁻Y⁺, and halogen, wherein R′ and R″ may independently be selected from optionally substituted C₁-C₂₀ alkyl; and Y is a base addition salt of the free carboxylate; and

ii) a glucosaminoglycan or a fragment or derivative thereof.

The term “optionally substituted” is intended to mean the substitution of one or more hydrogen atoms is substituted with another atom, chemical group or entity, termed substituents. Illustrative examples of substituents include carboxyl, formyl, amino, hydroxyl, halogen, nitro, sulphono, sulphanyl, C₁₋₆-alkyl, aryl, aryloxy, aryloxycarbonyl, arylcarbonyl, heteroaryl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkylsulphonyloxy, dihalogen-C₁₋₆-alkyl, trihalogen-C₁₋₆-alkyl, C₁₋₆-alkoxyl, oxo, C₁₋₆-carboxyl, C₁₋₆-alkoxycarbonyl, C₁₋₆-alkylcarbonyl, where aryl and heteroaryl representing substituents may be substituted 1-5 times with C₁₋₆-alkyl, C₁₋₆-alkoxy, nitro, cyano, hydroxy, amino or halogen. In general, the above substituents may be susceptible to further optional substitution.

The term C₁-C₂₀ alkyl is intended to mean a linear or branched saturated hydrocarbon chain wherein the longest chains has from one to twenty carbon atoms, such as methyl, ethyl, n-propyl, isopropyl, n-butyl, isobutyl, sec-butyl, tert-butyl, pentyl, isopentyl, neopentyl, hexyl, heptyl, octyl, undecacyl, dodecyl, etc. A branched hydrocarbon chain is intended to mean a C₁₋₂₀-alkyl substituted at any carbon with a hydrocarbon chain. The C₁-C₂₀ alkyl chain of the present invention may be optionally substituted.

The term “halogen” includes fluorine, chlorine, bromine and iodine.

It should also be understood that salts of compounds of formula 1 are anticipated, including, for instance hydrates and solvent addition forms. The term “base addition salts” include alkali metals, such as sodium and potassium, alkali earth metals, such as calcium and magnesium, and organic addition salts such as quaternary ammonium cations.

The chemical complex of the present invention relates to a complex obtainable from the combining of a pyridine carboxy derivative of Formula 1 and a glucosaminoglycan or a fragment or derivative thereof.

As stated, the complex comprises, in part, the optionally substituted pyridine carboxy derivative according to Formula 1 wherein R may be selected from OH; OR′; NH₂; NHR′; NR′R″, O⁻Y⁺, and halogen. R′ and R″ may independently be selected from optionally substituted C₁-C₂₀ alkyl.

The optionally substituted pyridine carboxy derivative may, for illustrative purposes, be selected from the group consisting of optionally substituted nicotinic acid, its corresponding acyl halide, ester, acid salt, or amide, nicotinamide; optionally substituted isonicotinic acid, its corresponding acyl halide, ester, acid salt, or amide, isonicotinamide; and optionally substituted picolinic acid, its corresponding acyl halide, ester, acid salt, or amide, picolinamide.

In the embodiment where the optionally substituted pyridine carboxy derivative is an amide, the amide may be its free primary amide (NH₂), its secondary amide (NHR′) or its tertiary amide (NR′R″).

As stated, the pyridine carboxy derivative may be optionally substituted. In one suitable embodiment, the pyridine carboxy is further substituted with a carboxy group such as a carboxylic acid, acyl halide, carboxylic ester, or acetamide. The pyridine carboxy may be substituted 0 to 4 times, such as 0, 1, 2, 3, or 4 times, preferably 0 to 1 time, most preferably 0 times.

In a preferred embodiment, the glucoaminoglycan is selected from the group comprising chondroitin sulfates, dermatan sulfates, keratan sulfates, hyaluronic acid and fragments and derivatives thereof.

In a suitable embodiment, the general structure of glucosaminoglycan chains comprise a linkage region, a chain cap and a repeat region.

In one aspect of the invention, the glucosaminoglycan consists of a repeat region, linkage region and a chain cap wherein the repeat region comprises at least one disaccharide unit in which one or both of the sugar monomers of said disaccharide unit is either galactosamine or glucosamine, such as N-acetylgalactosamine or N-acetylglucosamine; the linkage region is present at least once, is linked to the repeat region, may be a di-, oligo or poly-saccharide or a di-, oligo or poly-saccharide chain with a terminal amino acid; and is suitable for linking to a protein; and the cap region is a di-, oligo or poly-saccharide present at least once and is linked to the repeat region.

The repeat region comprises a repeating disaccharide. The repeat region consists of at least one disaccharide unit in which one or both of the sugar monomers of said disaccharide unit is either galactosamine or glucosamine, such as N-acetylgalactosamine or N-acetylglucosamine.

The other sugar monomer of said disaccharide repeating region may be selected from the array of hexoses known to the person skilled in the art. Illustrative examples of preferred embodiments of monomers include D-glucuronic acid, L-iduronic acid, D-galacturonic acid, D-galactose, and fucose, each of which may be optionally sulfonated or O-protected with a protective group known to the person skilled in the art.

The number of repeat units in a glucosaminoglycan chain may range from 1 to 500000, such as from 2 to 50000, preferably from 2 to 10000, most preferably from 2 to 1000. In suitable embodiments, the glucosaminoglycan comprises of 30 to 50 disaccharide units.

According to the invention preferred repeat regions may be selected from Formula 2, 3, 4, 5 ,6 ,7 or 8, which are non-limiting examples. Accordingly, the glucosaminoglycan chain may comprise of a repeat unit comprising of any combination of disaccharides according to Formula 2 to 8.

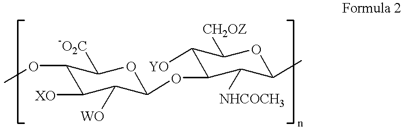

β-D-glucuronic acid N-acetyl-β-D-glucosamine, wherein W, X, Y, and Z independently may be SO₃⁻ or H.

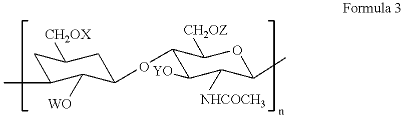

β-D-galactose N-acetyl-β-D-glucosamine, wherein W,X,Y and Z independently may be So₃⁻ or H.

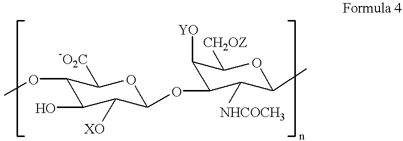

β-D-glucuronic acid N-acetyl-β-D-galactosamine, wherein X, Y and Z independently may be So₃⁻ or H.

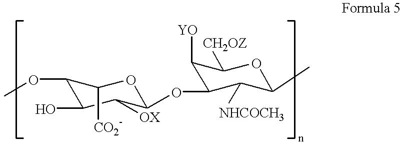

α-L-iduronic acid N-acetyl-β-D-galactosamine, wherein X, Y and Z may independently be SO₃⁻ or H.

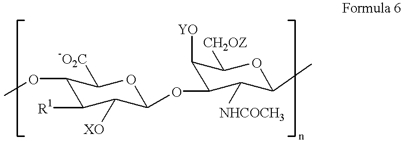

β-D-glucuronic acid N-acetyl-β-D-galactosamine, wherein R¹ may be fucose or SO₃⁻or OH and X, Y and Z independently may be SO₃⁻ or H.

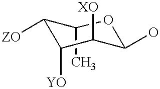

Fucose, wherein X, Y and Z independently may be SO₃⁻ or H.

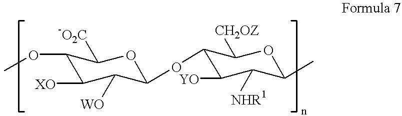

β-D-glucuronic acid D-glucosamine, wherein W, X, Y and Z independently may be SO₃⁻ or H and R′ may be COCH₃ or SO₃⁻.

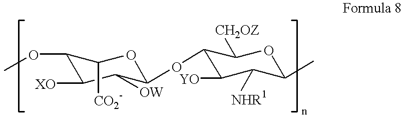

α-L-iduronic acid D-glucosamine, wherein W, X, Y and Z independently may be SO₃⁻ or H and R¹ may be COCH₃ or SO₃⁻.

As stated, the number of repeat units in a glucosaminoglycan chain may range from 1 to 500000. Accordingly the value of n, or the sum of n for all of the disaccharide units according the any of Formula 2 -8 (n₂+n₃+n₄+n₅+n₆+n₇+n₈), may be in the range of 1 to 500000, such as from 2 to 50000, preferably from 2 to 10000, most preferably from 2 to 1000. In suitable embodiments, the glucosaminoglycan comprises of 30 to 50 disaccharide units, i.e n may be in the range.

D-glucuronic acid is the principle uronic acid present in hyaluronic acid and chondroitin sulfates A and C, while dermatan sulfate (chondroitin sulfate B) contains L-iduronic acid. In keratan sulfate the principle uronic acid is D-galacturonic acid. In all of the glucosaminoglycans the disaccharide unit may be non-sulfated, mono-sulfated, di-sulfated or tri-sulfated, and different uronic acids may be present in a given polymer chain.

The linkage region is the moiety of the glucoaminoglycan which may be O-linked to a protein in the use of the chemical complex in the treatment defined herein. The linkage unit may be a di-, oligo or poly-saccharide and is linked to at least one repeat region. Linkage to a protein may be from any of the oxygen atoms of the terminal saccharide of the linkage unit. The linkage region may be linked to any part of the repreating region. Typically, the linkage region will be at the terminus of the repeating region.

Alternatively, the linkage region comprises a di-, oligo or poly-saccharide chain with a terminal amino acid. It such an embodiment, linkage to a protein is via said amino acid. In a preferred embodiment, the amino acid is serine.

A glucoaminoglycan may be linked more than once to a protein and there may be more than one linkage region. In a suitable embodiment, there is a one linkage region hence the glucoaminoglycan is linked once to a protein. Conversely, more than one glucoaminoglycan may be linked to a single protein.

In a preferred embodiment, the linkage region is of the general form: -4(GlcAβ(1-3)Galβ(1-3)Galβ(1-4)Xylβ(1-0)-Ser, where in C-4 and C-6 in Galβ independently may be SO₃ or OH. (Abbreviations: GlcA, glucuronic acid; Gal, galactose; Xyl, xylose; Ser, serine.)

In the embodiment wherein the glucoaminoglycan is chondroitin sulfate, said glucoaminoglycan is preferably O-linked to a serine of a protein core.

The chain cap is the chain terminus in the glucosaminoglycans. Thus, the chain cap comprises of a mono-, di- or oligo-saccharide at one or both of the two termini of the repeating regions of the glucosaminoglycans. In the typical embodiment wherein the linkage region is at the terminus of the repeat region, there will only be one chain cap.

The chain cap will typically be in the form of N-acetyl-β-D-galactosamine or N-acetyl-β-D-glucosamine, which may be non-sulfated, mono-sulfated or di-sulfated.

In a preferred embodiment, the chain cap will have a lower degree of sulfonation than the repeating region. The saccharide or saccharides of the chain may be O-protected in the manner known to the skilled artisan.

In the wherein the glucosaminoglycans is chondroitin sulfate, the chain cap may be in he form of formula 9.

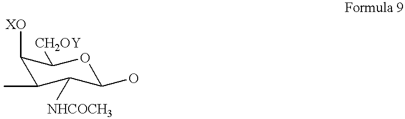

N-acetyl-β-D-galactosamine, wherein X and Y independently may be SO₃⁻ or OH.

Glucosaminoglycans, formerly known as mucopolysaccharides, are components of various tissues in numerous animals, both vertebrates and invertebrates. All are polymers of repeating disaccharide units, in which one of the sugars is either N-acetylgalactosamine or N-acetylglucosamine.

Important examples are the hyaluronic acid, chondroitin sulfates and keratan sulfates of connective tissue, the dermatan sulfates (chondroitin sulfates B) of skin, and heparin and heparan sulfates of mast cells.

A major function of some glycosaminoglycans is the formation of a matrix to hold together the protein components of the skin and connective tissue.

The filamentous structure is built on a single hyaluronic acid molecule, to which extended core proteins are attached noncovalently. These, in turn, have chondroitin sulfate and keratan sulfate chains covalently bound to them though serine side chains. In cartilage, this kind of structure binds collagen and helps hold the collagen fibres in a tight, strong network. The binding apparently involves electrostatic interactions between the sulfate and/or carboxylate groups and basic side chains in collagen.

The glucosaminoglucans of the present invention may be obtained from any such biological glucosaminoglycan-containing material by any available method obvious to a person skilled in the art, e.g. chemical and/or mechanical processing. Thus according to the invention glycosaminglycan polymers can be depolymerised, for example by a number of specific enzymes or by acid hydrolysis to give low molecular weight species, which carry a range of negative charges depending on the number of sulphated groups attached. A number of relevant enzymes are available such as chondroitinases, keratanases, hyaluronidases, heparinases, heparitinases etc. There are a number of subtypes of such enzymes with selective activity making it possible to manufacture numerous fragments of glucosaminoglycans, which can be used according to the invention. The molecular weight of such fragments is preferably in the range from 5000 to 1000000 Da, e.g. from 6000 to 500000 Da, such as from 7000 to 300000 Da, even more preferably from 8000 to 200000 Da, such as from 9000 to 100000 Da, e.g. from 10000 to 50000 Da. In another preferred embodiment of the invention the molecular weight of the glucosaminoglucan fragment is below 5000 Da and even more preferably below 3000 Da.

The depolymerisation of the naturally occurring (native) glycosaminoglycan polymers leads to a sequence of fragments of varying size depending on the degree of hydrolysis. These will, according to the invention, be a mixture of fragments composed of repeat units, fragments with the original chain cap and fragments with the original linkage region linked to a peptide or protein. Thus non-limiting examples of such fragments may be any combination of formula 2 to 9, the linkage region and the chain cap.

According to the invention the percentage of protein in a mixture of glycosaminoglycan fragments used in a complex of the invention may be from 0 to 50 %, such as from 0 to 25 %, e.g. from 0 to 20 %, such as from 0 to 15 %, e.g. from 0 to 10 %, such as from 0 to 5 %, e.g. from 0 to 4 %, such as from 0 to 3 %, e.g. from 0 to 2 %, such as from 0 to 1%.

According to the invention the molecular size of such fragments or derivatives of glucosaminoglycans may be any size obtainable through processing of natural glucosaminoglycan containing material or by chemical synthesis. In a preferred embodiment of the invention the molecular weight of such fragments is in the range from 5000 to 1000000 Da, e.g. from 6000 to 500000 Da, such as from 7000 to 300000 Da, even more preferably from 8000 to 200000 Da, such as from 9000 to 100000 Da, e.g. from 10000 to 50000 Da. In another preferred embodiment of the invention the molecular weight of the glucosaminoglucan fragment is below 5000 Da and even more preferably below 3000 Da.

Surprisingly, it has further been found by the present investigators that small fragments of glucosaminoglucans with a molecular weight below 5000 Da have a very high pharmacological activity and therefore may be used alone to treat hypersensitivity, inflammation, etc. according to the invention, besides being used as components of the complexes according to the invention. These small fragments may be monomers, dimers, trimers, tetramers or pentamers of the disaccharide building blocks of glucosaminoglycans. Non-limiting examples of such small fragments are monomers, dimers, trimers, tetramers or pentamers of the structures shown in formula 2, 3, 4, 5 ,6 7 and 8 or combinations thereof with chain caps or linkage regions as defined above.

Glucosaminoglycan fragments of the invention may be produced from a natural source or by chemical synthesis. Thus according to the invention glucosaminoglycans may be obtained from any natural source, plant or animal, vertebrates or invertebrates. For example chondroitin sulfate may be obtained from the cartilage of fish, especially sharks and the cartilage of land animals, e.g. pig, ox and chicken. Also, the fragments may be conjugated or chemically modified to obtain derivatives of glucosaminoglycans with certain desirable properties. Thus any known method of chemical modification or synthesis may be applied, which would be obvious to a person skilled in the art.

The glycosaminoglycan polymers or fragments or derivatives thereof according to the invention are negatively charged and are found as salts soluble in water. According to the invention any positively charged ion may be the component of such salts. In a preferred embodiment of the invention the positively charged ion is: Na⁺, K⁺, NH₄⁺, Mg²⁺ or Ca²⁺. Zi²⁺, Fe²⁺.

Due to the complexity of glucosaminoglycans or fragments or derivatives thereof, they may be difficult to characterise chemically. In the case of chondroitin sulfates an official monograph has been proposed, which is given in the following.

According to Pharmacopial Forum, vol. 26(5) [September-October 2000] p. 1432-1434 test methods for Identification, purity, content of protein and content of glucosaminoglycans (mucopolysaccharides) are described for Chondroitin 4-sulfate Sodium salt and Chondroitin 6-sulfate Sodium salt using USP Chondroitin sulfate Sodium RS, as reference.

The methods/tests are as follows:

{11} USP Chondroitin Sulfate Sodium RS—Dry portion at 105° C. for 4 hours just before use. [Note-This material is extremely hygroscopic once dried. Weigh promptly, avoiding moisture from the environment]. Keep container tightly closed.

Identification

A: infrared Absorption {197K}

B: The residue, as obtained from the test for Residue on ignition, is soluble in water and meets the requirements on the test for Sodium {191} and Sulfate {191}.

Clarity and color of solution

Transfer 2.5g of Chondroitin Sulfate Sodium to 50.ml volumetric flask. Dissolve in and dilute with carbon dioxide-free water to volume, mix, and examine immediately. Measure the absorbance of this solution at 420 nm in a 1-cm cell, using carbon dioxide-free water as the blank: its absorbance is not more than 0,35.

Specific rotation {781S}

Between −20.0° and −30.0°, test solution 30mg per ml.

Microbial limits {2021}

The total bacterial count does not exceed 1000 per g, the total combined molds and yeasts count does not exceed 100 per g, and it meets the requirements of the tests for absence of Clostridium species, Salmonella species, and Escherichia coli.

Ph {791}

Between 5.5 and 7.5 in a solution (1 in 100).

Loss on drying {731}

Dry it at 105° C. for 4 hours: it loses not more than 10% of its weight. [Note-chondroitin sulfate sodium is extremely hydroscopic once dried. Avoid exposure to the atmosphere, and weigh promptly.]

Residue on ignition {281}

Between 20.0% and 30.0% on the dried basis, omitting the addition of sulphuric acid. [Note-Retain a portion of the residue for Identification test B.]

Chloride {221}

A 0.10g portion shows no more chloride than corresponds to 0.4mi of 0.020N hydrochloric acid: not more than 0.50% is found.

Sulfate {221}

A 0.10g portion shows no more sulfate than corresponds to 0.25mi 0.020 N sulphuric acid: not more than 0.24% is found.

Arsenic, Method II {221}

Not more than 2μg per g.

Heavy metals, Method II {231}

Not more than 0.002%.

Organic volatile impurities, Method I {467}

Meets the requirements.

Electrophoretic purity

Not yet defined

Content of protein

Standard solution—Transfer an accurately measured volume of 7 percent Bovine Serum Albumin Certified Standard to a suitable container, and dilute quantitatively and stepwise with water to obtain a solution having a known concentration of about 35 μg per ml.

Test solution—Transfer an accurately weighed amount of Chondroitin Sulfate Sodium, equivalent to 100mg of the dried substance, to a 100mg volumetric flask, dissolve in and dilute with water to volume, and mix.

Procedure—Add 2.0ml of freshly prepared alkaline cupric tartrate TS to test tubes containing 2.0ml of water, 2.0ml of the test solution, or 2.0ml of the Standard solution. Mix after each addition. After about 10 minutes, add to each test tube 1.0ml of Folin-Ciocalteu Phenol TS, prepared immediately before use, and mix. After 30 minutes, measure the absorbance of each solution at 750nm against blank. The absorbance of the test solution is not more than the absorbance of the Standard solution: not more than 3.5% of proteins are found.

Content of total mucopolysaccharides

Standard solution—Tansfer about 25mg of USP Chondroitin Sulfate Sodium RS, accurately weighed, to a 25ml volumetric flask. [Note-Chondroitin Sulfate Sodium is extremely hydroscopic once dried. Avoid exposure to the atmosphere, and weigh promptly]. Dissolve in 6 ml water, add 1ml of pH 7.2 phosphate buffer solution, and dilute with water to volume to obtain a solution having a known concentrations of about 1 mg per ml.

Test solution—Transfer about 100mg of Chondroitn Sulfate Sodium, accurately weighed, to a 100ml volumetric flask, dissolve in 30ml of water, add 5ml of pH 7.2 phosphate buffer solution dilute with water to volume, and mix.

Cetyl pyridinium chloride solution—Perpare a solution of cetylpyridinium chloride in water having a concentration of about 1 mg per ml.

Procedure—Transfer 5 ml of Standard solution to the titration vessel, and add about 30ml of water. Titrate with Cetyl pyridinium chloride solution using a phototrode to determine the endpoint turbidimetrically, at 420nm, with the instrument set in transmittance mode at 70% for the initial solution. Perform a blank determination, and make any necessary correction. Determine the equivalence factor, F, in mg of USP Chondroitin Sulfate Sodium RS per each ml of Cetyl pyridinium chloride solution, taken by the formula:

5(C/Vₛ)

in which C is the concentration, in mg per ml, of USP Chondroitin Sulfate Sodium RS in the Standard solution; and Vₛ is the volume, in ml, of Cetyl pyridinium chloride solution consumed by the Standard solution. Repeat this procedure, except to use 5ml of the Test solution instead of the Standard solution. Calculate the percentage of total mucopolysaccharide as Chondrotin sulfate Sodium in the portion of Chondroitin Sulfate Sodium taken by the formula:

2000F(Vᵤ/W),

in which F is the equivalence factor, in mg per ml, calculated above; Vᵤ is the volume, in ml, of Cetyl pyridinium chloride solution consumed by the Test solution; and W is the weight, in mg, of the portion of Chondroitin Sulfate Sodium taken.

These methods according to Pharmacopial Forum may also be applied to other glucosaminoglycans than chondroitin sulfate, with or without modifications.

Accordingly, the present invention provides a chemical complex, a pharmaceutical composition, a dietary supplement or a cosmetic comprising:

i) an optionally substituted pyridine carboxy derivativeaccording to formula 1

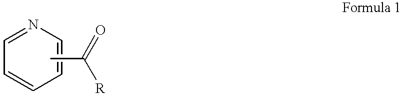

wherein R may be selected from OH; OR′; NH₂; NHR′; NR′R″, O⁻Y⁺, and halogen, wherein R′ and R″ may independently be selected from optionally substituted C₁-C₂₀ alkyl; and Y is a base addition salt of the free carboxylate; and

ii) a glucosaminoglycan or a fragment or derivative thereof; and optionally

iii) a pharmaceutically acceptable carrier, wherein p the molar ratio between the pyridine carboxy derivative and the glucosaminoglycan is in the range from 1:100000 to 100000:1, e.g. from 1:50000 to 50000:1, such as from 1:25000 to 25000: 1, such as from 1:15000 to 15000: 1, preferably from 1:10000 to 10000:1, e.g. from 1:8000 to 8000:1, such as from 1:6000 to 6000:1, more preferably in the range from 1:5000 to 5000:1, e.g. from 1:4000 to 4000:1, such as from 1:3000 to 3000:1, e.g. from 1:2000 to 2000:1, e.g. from 1:1000 to 1000:1, such as from 1:900 to 900:1, e.g. from 1:800 to 800:1, such as from 1:700 to 700:1, e.g. from 1:600 to 600:1, e.g. from 1:500 to 500:1, such as from 1:400 to 400:1, e.g. from 1:300 to 300: 1, such as from 1:200 to 200: 1, e.g. from 1:100 to 100: 1, such as from 1:90 to 90:1, e.g. from 1:80 to 80:1, such as from 1:70 to 70:1, e.g. from 1:60 to 60:1, e.g. from 1:50 to 50:1, such as from 1:40 to 40:1, e.g. from 1:30 to 30:1, such as from 1:20 to 20:1, e.g. from 1:10 to 10:1, such as 1:5 to 5:1, e.g.1:1.

In a preferred embodiment of the invention the pyridine carboxy derivative is selected from the group consisting of nicotinamide and isonicotinamide, most preferably nicotinamide (niacinamide).

According to the invention, the above mentioned chemical complexes or compositions can be combined with any other active ingredient to potentiate the therapeutic action.

A “dietary supplement” is defined according to the U.S. Food and Drug Administration in the Dietary Supplement Health and Education Act of 1994 (DSHEA).

The DSHEA gives the following formal definition of a “dietary supplement”: “A dietary supplement:

is a product (other than tobacco) that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract, or combinations of these things.

is intended for ingestion in pill, capsule, tablet, or liquid form.”

Similar definitions exist in other parts of the world, e.g. in Europe; in the present context, the definition is as defined above. Different denominations concerning “dietary supplements” are used around the world, such as “food supplements”, “neutraceuticals”, “functional foods” or simply “foods”. In the present context the term “dietary supplement” covers any such denomination or definition.

“Systemic administration” is defined as administration by the parenteral route such as the intravenous, intraperitoneal, intraarticular, intraventricular, intracapsular, intraspinal, intramuscular, subcutaneous, intradermal, oral, buccal, sublingual, nasal, rectal, vaginal or transdermal routes.

“Topical administration” is used in its conventional sense to mean delivery of a topical chemical complex or pharmacologically active composition to the skin or mucosa.

The above mentioned pharmacological actions provide part of the rationale for the following therapeutic applications of a chemical complex or a composition comprising an optionally substituted pyridine carboxy derivative and a glucosaminoglycan and optionally a pharmaceutically acceptable carrier:

A method for the treatment or prevention of hypersensitivity disease or inflammation characterised by the administration of the above mentioned chemical complexes or compositions to a mammal, preferentially a human. The therapeutic action may be relevant to all known diseases associated with hypersensitivity reactions or inflammation. Autoimmune disorders and IgE mediated allergic conditions are described below in more detail. Besides these specific therapeutic areas, the action of the above mentioned composition is relevant to all known conditions and diseases associated with hypersensitivity reaction, and the following examples are not limiting with respect to this: infections (viral, bacterial, fungal, parasitic, etc.), cold and flu, contact dermatitis, insect bites, allergic vasculitis, postoperative reactions, transplantation rejection (graft-versus-host disease), etc.

A method for the treatment or prevention of autoimmune disorders characterised by the administration of the above mentioned chemical complexes or compositions to a mammal, preferentially a human. The applicant puts forward the hypothesis that the therapeutic action is due to the immunomodulating and suppressing effect on hypersensitivity reactions of the above mentioned chemical complex or composition. The therapeutic action may be relevant to all known autoimmune disorders and the following examples are not limiting with respect to this: Autoimmune hepatitis, Primary biliary cirrhosis, Primary sclerosing cholangitis, Autoimmune hemolytic anemias, Grave's disease, Myasthenia gravis, Type 1 Diabetes Mellitus, Inflammatory myopathies, Multiple sclerosis, Hashimoto's thyreoiditis, Autoimmune adrenalitis, Crohn's Disease, Ulcerative Colitis, Glomerulonephritis, Progressive Systemic Sclerosis (Scleroderma), Sjögren's Disease, Lupus Erythematosus, Primary vasculitis, Rheumatoid Arthritis, Juvenile Arthritis, Mixed Connective Tissue Disease, Psoriasis, Pemfigus, Pemfigoid, Dermatitis Herpetiformis, etc.

A method for the treatment or prevention of an IgE mediated allergic reaction or condition characterised by the administration of the above mentioned chemical complexes or compositions to a mammal, preferentially a human. The applicant puts forward the hypothesis that the therapeutic action is due to the suppressing effect on hypersensitivity reaction of the above mentioned compositions. The therapeutic action may be relevant to all known IgE mediated allergic reactions and conditions, and the following examples are not limiting with respect to this: asthma, eczema (e.g. atopic dermatitis), urticaria, allergic rhinitis, anaphylaxis, etc.

A method for the treatment or prevention of any condition associated with pain characterised by the administration of the above mentioned chemical complexes or compositions to a mammal, preferentially a human. The applicant puts forward the hypothesis that the therapeutic action is related to immunomodulation, possibly to a suppressing effect on hypersensitivity reactions.

A method for chondroprotection or regeneration of articular cartilage. This may be relevant for the treatment or prophylaxis of any type of arthritis, especially osteoarthritis or for the general promotion of joint health in a mammal, preferentially in a human.

According to the invention the above mentioned chemical complexes or compositions can be combined with any other active ingredients to potentiate the therapeutic action.

In a preferred embodiment of the invention the above mentioned chemical complexes or compositions are used for systemic administration.

In another preferred embodiment of the invention the above mentioned chemical complexes or compositions are used for topical administration.

A pharmaceutical acceptable carrier for systemic or topical administration can be water or vehicles other than water, said other vehicles can be used in the compositions and can include solids or liquids such as emollients, solvents, humectants, thickeners and powders. Examples of each of these types of vehicles, which can be used singly or as compositions of one or more vehicles, are as follows:

Emollients, such as stearyl alcohol, glyceryl monoricinoleate, glyceryl monostearate, propane-1,2-diol, butane-1,3-diol, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polyethylene glycol, triethylene glycol, lanolin, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate;

solvents, such as water, methylene chloride, isopropanol, castor oil, ethylene glycol monoethyl ether, diethylene glycol monobutyl ether, diethylene glycol monoethyl ether, dimethyl sulfoxide, tetrahydrofuran, vegetable and animal oils, glycerol, ethanol, propanol, propylene glycol, and other glycols or alcohols, fixed oils;

humectants or moistening agents, such as glycerin, sorbitol, sodium 2-pyrrolidone-5-carboxylate, soluble collagen, dibutyl phthalate, gelatin; powders, such as chalk, talc, kaolin, starch and derivatives thereof, gums, colloidal silicon dioxide, sodium polyacrylate, chemically modified magnesium aluminium silicate, hydrated aluminium silicate, carboxyvinyl polymer, sodium carboxymethyl cellulose, ethylene glycol monostearate;

gelling or swelling agents, such as pectin, gelatin and derivatives thereof, cellulose derivatives such as methyl cellulose, carboxymethyl cellulose or oxidised cellulose, cellulose gum, guar gum, acacia gum, karaya gum, tragacanth gum, bentonite, agar, alginates, carbomer, gelatine, bladderwrack, ceratonia, dextran and derivatives thereof, ghatti gum, hectorite, ispaghula husk, xanthan gum;

polymers, such as polylactic acid or polyglycolic acid polymers or copolymers thereof, paraffin, polyethylene, polyethylene oxide, polyethylene glycol, polypropylene glycol, polyvinylpyrrolidone;

surfactants, such as non-ionic surfactants, e.g. glycol and glycerol esters, macrogol ethers and esters, sugar ethers and esters, such as sorbitan esters, ionic surfactants, such as amine soaps, metallic soaps, sulfated fatty alcohols, alkyl ether sulfates, sulfated oils, and ampholytic surfactants and lecitins;

buffering agents, such as sodium, potassium, aluminium, magnesium or calcium salts (such as the chloride, carbonate, bicarbonate, citrate, gluconate, lactate, acetate, gluceptate or tartrate).

The active ingredients of the chemical complex or pharmaceutical composition of the present invention need not be administered as one pharmaceutically entity, but can of course be administered as individual compounds or pharmaceutical compositions, i.e. as

ia) an optionally substituted pyridine carboxy derivative according to formula 1

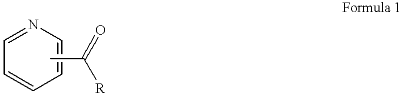

wherein R may be selected from OH; OR′; NH₂; NHR′; NR′R″, O⁻Y⁺, and halogen, wherein R′ and R″ may independently be selected from optionally substituted C₁-C₂₀ alkyl; and Y is a base addition salt of the free carboxylate; and

optionally with iia) a pharmaceutically acceptable carrier as one component as the one pharmaceutically entity, and

ib) a glucosaminoglycan or a fragment or derivative thereof; and optionally iib) a pharmaceutically acceptable carrier as the second pharmaceutically entity.

Furthermore, it is obvious that in the use according to the invention for the preparation of medicaments or dietary supplements, the above mentioned compositions may be mixed with additives such as surfactants, solvents, thickeners, stabilisers, preservatives, antioxidants, flavours, etc. to obtain a desirable product formulation suitable for systemic administration. Similarly, a pharmaceutical or dietary supplement according to the invention may further contain such additives. There are no limitations on the route of administration or dosage form of the formulation, and the following examples are not limiting with respect to this: tablets, capsules, lozenges, chewing gum, fluids, granulates, sprays (e.g. aerosol), inhalants, etc.

Optionally, the composition may also contain surfactants such as bile salts, polyoxyethylene-sorbitan-fatty acid esters or polyalcohol mixed chain-length fatty acid esters for improving dispersibility of the composition in the digestive fluids leading to improved bioavailability or for obtaining the final dosage form of the composition.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver compositions of the invention. Additionally, the compositions may be delivered using a sustained-release system, such as semi-permeable matrices of solid polymers containing the therapeutic agent. Various sustained-release materials have been established and are well known by those skilled in the art. Sustained-release capsules may, depending on their chemical nature, release the compositions for a few weeks up to over 100 days.

Furthermore, the invention relates to a method for the preparation of a chemical complex or a pharmaceutically active composition as described above characterised by obtaining a glucosaminoglycan or fragment or derivative thereof, and pyridine carboxy derivative according to formula 1 as described above; and mixing said glucosaminglycan and pyridine carboxy derivative, optionally with a pharmaceutically acceptable carrier.

### EXAMPLES

The following examples 1-133 describe the preparation of chemical complexes of the present invention.

### General method example 1-108

The glucosaminoglycans are hygroscopic and are dried before use. The glucosaminoglycan and the pyridine carboxy derivative are dissolved in as little water as possible and the solvent is removed by spray drying or freeze-drying. After the solvent is removed the product is a white to yellowish hygroscopic powder.

The powder is suitable for any type of product e.g. pharmaceutical products, dietary supplements and cosmetic formulations. Non-limiting examples of such products are tablets, capsules, ointments and lotions as described above.

### Example 1 to 44

### Molar ratio glucosaminoglycan/pyridine carboxy derivative 1: 1000 (mol/mol).

| | | | | Average |
|---|---|---|---|---|
| | | | Origin | molecular |
| | Pyridine | | of the | weight of the |
| | carboxy | Glucosamino- | glucos- | glucos- |
| | derivative | glycan | amino- | aminoglycan |
| | (quantity) | (quantity) | glycan | (g/mol) |
| Example 1. | Niacinamide | Chondroitin sulfate | Bovine | 50,000 |
| | (2442 g) | (1000 g) | | |
| Example 2. | Niacinamide | Chondroitin sulfate | Porcine | 50,000 |
| | (2442 g) | (1000 g) | | |
| Example 3. | Niacinamide | Chondroitin sulfate | Shark | 50,000 |
| | (2442 g) | (1000 g) | | |
| Example 4. | Niacinamide | Chondroitin sulfate | Bovine | 17,000 |
| | (7182 g) | (1000 g) | | |
| Example 5. | Niacinamide | Chondroitin sulfate | Porcine | 17,000 |
| | (7182 g) | (1000 g) | | |
| Example 6. | Niacinamide | Chondroitin sulfate | Shark | 17,000 |
| | (7182 g) | (1000 g) | | |
| Example 7. | Niacinamide | Chondroitin sulfate | Bovine | 4,000 |
| | (30,525 g) | (1000 g) | | |
| Example 8. | Niacinamide | Chondroitin sulfate | Porcine | 4,000 |
| | (30,525 g) | (1000 g) | | |
| Example 9. | Niacinamide | Chondroitin sulfate | Shark | 4,000 |
| | (30,525 g) | (1000 g) | | |
| Example 10. | Niacinamide | Hyaluronate | Bovine | 50,000 |
| | (2442 g) | (1000 g) | | |
| Example 11. | Niacinamide | Hyaluronate | Porcine | 50,000 |
| | (2442 g) | (1000 g) | | |
| Example 12. | Niacinamide | Hyaluronate | Shark | 50,000 |
| | (2442 g) | (1000 g) | | |
| Example 13. | Niacinamide | Hyaluronate | Bovine | 17,000 |
| | (7184 g) | (1000 g) | | |
| Example 14. | Niacinamide | Hyaluronate | Porcine | 17,000 |
| | (7182 g) | (1000 g) | | |
| Example 15. | Niacinamide | Hyaluronate | Shark | 17,000 |
| | (7182 g) | (1000 g) | | |
| Example 16. | Niacinamide | Hyaluronate | Bovine | 4,000 |
| | (30,525 g) | (1000 g) | | |
| Example 17. | Niacinamide | Hyaluronate | Porcine | 4,000 |
| | (30,525 g) | (1000 g) | | |
| Example 18. | Niacinamide | Hyaluronate | Shark | 4,000 |
| | (30,525 g) | (1000 g) | | |
| Example 19. | Niacinamide | Keratan sulfate | Bovine | 50,000 |
| | (2442 g) | (1000 g) | | |
| Example 20. | Niacinamide | Keratan sulfate | Porcine | 50,000 |
| | (2442 g) | (1000 g) | | |
| Example 21. | Niacinamide | Keratan sulfate | Shark | 50,000 |
| | (2442 g) | (1000 g) | | |
| Example 22. | Niacinamide | Keratan sulfate | Bovine | 17,000 |
| | (7184 g) | (1000 g) | | |
| Example 23. | Niacinamide | Keratan sulfate | Porcine | 17,000 |
| | (7182 g) | (1000 g) | | |
| Example 24. | Niacinamide | Keratan sulfate | Shark | 17,000 |
| | (7182 g) | (1000 g) | | |
| Example 25. | Niacinamide | Keratan sulfate | Bovine | 4,000 |
| | (30,525 g) | (1000 g) | | |
| Example 26. | Niacinamide | Keratan sulfate | Porcine | 4,000 |
| | (30,525 g) | (1000 g) | | |
| Example 27. | Niacinamide | Keratan sulfate | Shark | 4,000 |
| | (30,525 g) | (1000 g) | | |
| Example 28. | Niacinamide | Dermatan sulfate | Bovine | 50,000 |
| | (2442 g) | (1000 g) | | |
| Example 29. | Niacinamide | Dermatan sulfate | Porcine | 50,000 |
| | (2442 g) | (1000 g) | | |
| Example 30. | Niacinamide | Dermatan sulfate | Shark | 50,000 |
| | (2442 g) | (1000 g) | | |
| Example 31. | Niacinamide | Dermatan sulfate | Bovine | 17,000 |
| | (7184 g) | (1000 g) | | |
| Example 32. | Niacinamide | Dermatan sulfate | Porcine | 17,000 |
| | (7182 g) | (1000 g) | | |
| Example 33. | Niacinamide | Dermatan sulfate | Shark | 17,000 |
| | (7182 g) | (1000 g) | | |
| Example 34. | Niacinamide | Dermatan sulfate | Bovine | 4,000 |
| | (30,525 g) | (1000 g) | | |
| Example 35. | Niacinamide | Dermatan sulfate | Porcine | 4,000 |
| | (30,525 g) | (1000 g) | | |
| Example 36. | Niacinamide | Dermatan sulfate | Shark | 4,000 |
| | (30,525 g) | (1000 g) | | |
| Example 37. | N2-methyl- | Chondroitin sulfate | Bovine | 50,000 |
| | niacinamide | (1000 g) | | |
| | (2722 g) | | | |
| Example 38. | N2-methyl- | Chondroitin sulfate | Porcine | 50,000 |
| | niacinamide | (1000 g) | | |
| | (2722 g) | | | |
| Example 39. | N2-methyl- | Chondroitin sulfate | Bovine | 4,000 |
| | niacinamide | (1000 g) | | |
| | (34,025 g) | | | |
| Example 40. | N2-ethyl- | Chondroitin sulfate | Bovine | 50,000 |
| | niacinamide | (1000 g) | | |
| | (3002 g) | | | |
| Example 41. | N2-ethyl- | Chondroitin sulfate | Porcine | 50,000 |
| | niacinamide | (1000 g) | | |
| | (3002 g) | | | |
| Example 42. | N2-ethyl- | Chondroitin sulfate | Bovine | 4,000 |
| | niacinamide | (1000 g) | | |
| | (37,525 g) | | | |
| Example 43. | N2-ethyl- | Chondroitin sulfate | Bovine | 17,000 |
| | niacinamide | (1000 g) | | |
| | (8824 g) | | | |
| Example 44. | Niacinamide | Chondroitin sulfate | Porcine | 30,000 |
| | (4070 g) | (1000 g) | | |

### Example 45 to 48

### Molar ratio glucosaminoglycan/pyridine carboxy derivative 1:500 (mol/mol).

| | | | | Average |
|---|---|---|---|---|
| | | | Origin | molecular |
| | Pyridine | | of the | weight of the |
| | carboxy | Glucosamino- | glucos- | glucos- |
| | derivative | glycan | amino- | aminoglycan |
| | (quantity) | (quantity) | glycan | (g/mol) |
| Example 45. | Niacinamide | Chondroitin sulfate | Bovine | 17,000 |
| | (3591 g) | (1000 g) | | |
| Example 46. | Niacinamide | Chondroitin sulfate | Porcine | 30,000 |
| | (2035 g) | (1000 g) | | |
| Example 47. | Niacinamide | Heparan sulfate | Bovine | 7,500 |
| | (8140 g) | (1000 g) | | |
| Example 48. | N2-ethyl- | Chondroitin sulfate | Bovine | 17,000 |
| | niacinamide | (1000 g) | | |
| | (4412 g) | | | |

### Example 49 to 52

### Molar ratio glycosaminoglycan/pyridine carboxy derivative 1:200 (mol/mol).

| | | | | Average |
|---|---|---|---|---|
| | | | Origin | molecular |
| | Pyridine | | of the | weight of the |
| | carboxy | Glucosamino- | glucos- | glucos- |
| | derivative | glycan | amino- | aminoglycan |
| | (quantity) | (quantity) | glycan | (g/mol) |
| Example 49. | Niacinamide | Chondroitin sulfate | Bovine | 17,000 |
| | (1436 g) | (1000 g) | | |
| Example 50. | Niacinamide | Chondroitin sulfate | Porcine | 30,000 |
| | (814 g) | (1000 g) | | |
| Example 51. | Niacinamide | Heparin (1000 g) | Porcine | 6,000 |
| | (4070 g) | | | |
| Example 52. | N2-ethyl- | Chondroitin sulfate | Bovine | 17,000 |
| | niacinamide | (1000 g) | | |
| | (1765 g) | | | |

### Example 53 to 54

### Molar ratio glucosaminoglycan/pyridine carboxy derivative 1:100 (mol/mol).

| | | | | Average |
|---|---|---|---|---|
| | | | Origin | molecular |
| | Pyridine | | of the | weight of the |
| | carboxy | Glucosamino- | glucos- | glucos- |
| | derivative | glycan | amino- | aminoglycan |
| | (quantity) | (quantity) | glycan | (g/mol) |
| Example 53. | Niacinamide | Heparin (1000 g) | Porcine | 6,000 |
| | (2035 g) | | | |
| Example 54. | Niacinamide | Heparan sulfate | Bovine | 7,500 |
| | (1628 g) | (1000 g) | | |

### Example 55 to 92

### Molar ratio glucosaminoglycan/pyridine carboxy derivative 1:50 (mol/mol).

| | | | | Average |
|---|---|---|---|---|
| | | | Origin | molecular |
| | Pyridine | | of the | weight of the |
| | carboxy | Glucosamino- | glucos- | glucos- |
| | derivative | glycan | amino- | aminoglycan |
| | (quantity) | (quantity) | glycan | (g/mol) |
| Example 55. | Niacinamide | Chondroitin sulfate | Bovine | 50,000 |
| | (122 g) | (1000 g) | | |
| Example 56. | Niacinamide | Chondroitin sulfate | Porcine | 50,000 |
| | (122 g) | (1000 g) | | |
| Example 57. | Niacinamide | Chondroitin sulfate | Shark | 50,000 |
| | (122 g) | (1000 g) | | |
| Example 58. | Niacinamide | Chondroitin sulfate | Bovine | 17,000 |
| | (359 g) | (1000 g) | | |
| Example 59. | Niacinamide | Chondroitin sulfate | Porcine | 17,000 |
| | (359 g) | (1000 g) | | |
| Example 60. | Niacinamide | Chondroitin sulfate | Shark | 17,000 |
| | (359 g) | (1000 g) | | |
| Example 61. | Niacinamide | Chondroitin sulfate | Bovine | 4,000 |
| | (1526 g) | (1000 g) | | |
| Example 62. | Niacinamide | Chondroitin sulfate | Porcine | 4,000 |
| | (1526 g) | (1000 g) | | |
| Example 63. | Niacinamide | Chondroitin sulfate | Shark | 4,000 |
| | (1526 g) | (1000 g) | | |
| Example 64. | Niacinamide | Hyaluronate | Bovine | 50,000 |
| | (122 g) | (1000 g) | | |
| Example 65. | Niacinamide | Hyaluronate | Porcine | 50,000 |
| | (122 g) | (1000 g) | | |
| Example 66. | Niacinamide | Hyaluronate | Shark | 50,000 |
| | (122 g) | (1000 g) | | |
| Example 67. | Niacinamide | Hyaluronate | Bovine | 17,000 |
| | (359 g) | (1000 g) | | |
| Example 68. | Niacinamide | Hyaluronate | Porcine | 17,000 |
| | (359 g) | (1000 g) | | |
| Example 69. | Niacinamide | Hyaluronate | Shark | 17,000 |
| | (359 g) | (1000 g) | | |
| Example 70. | Niacinamide | Hyaluronate | Bovine | 4,000 |
| | (1526 g) | (1000 g) | | |
| Example 71. | Niacinamide | Hyaluronate | Porcine | 4,000 |
| | (1526 g) | (1000 g) | | |
| Example 72. | Niacinamide | Hyaluronate | Shark | 4,000 |
| | (1526 g) | (1000 g) | | |
| Example 73. | Niacinamide | Keratan sulfate | Bovine | 50,000 |
| | (122 g) | (1000 g) | | |
| Example 74. | Niacinamide | Keratan sulfate | Porcine | 50,000 |
| | (122 g) | (1000 g) | | |
| Example 75. | Niacinamide | Keratan sulfate | Shark | 50,000 |
| | (122 g) | (1000 g) | | |
| Example 76. | Niacinamide | Keratan sulfate | Bovine | 17,000 |
| | (359 g) | (1000 g) | | |
| Example 77. | Niacinamide | Keratan sulfate | Porcine | 17,000 |
| | (359 g) | (1000 g) | | |
| Example 78. | Niacinamide | Keratan sulfate | Shark | 17,000 |
| | (359 g) | (1000 g) | | |
| Example 79. | Niacinamide | Keratan sulfate | Bovine | 4,000 |
| | (1526 g) | (1000 g) | | |
| Example 80. | Niacinamide | Keratan sulfate | Porcine | 4,000 |
| | (1526 g) | (1000 g) | | |
| Example 81. | Niacinamide | Keratan sulfate | Shark | 4,000 |
| | (1526 g) | (1000 g) | | |
| Example 82. | Niacinamide | Dermatan sulfate | Bovine | 50,000 |
| | (122 g) | (1000 g) | | |
| Example 83. | Niacinamide | Dermatan sulfate | Porcine | 50,000 |
| | (122 g) | (1000 g) | | |
| Example 84. | Niacinamide | Dermatan sulfate | Shark | 50,000 |
| | (122 g) | (1000 g) | | |
| Example 85. | Niacinamide | Dermatan sulfate | Bovine | 17,000 |
| | (359 g) | (1000 g) | | |
| Example 86. | Niacinamide | Dermatan sulfate | Porcine | 17,000 |
| | (359 g) | (1000 g) | | |
| Example 87. | Niacinamide | Dermatan sulfate | Shark | 17,000 |
| | (359 g) | (1000 g) | | |
| Example 88. | Niacinamide | Dermatan sulfate | Bovine | 4,000 |
| | (1526 g) | (1000 g) | | |
| Example 89. | Niacinamide | Dermatan sulfate | Porcine | 4,000 |
| | (1526 g) | (1000 g) | | |
| Example 90. | Niacinamide | Dermatan sulfate | Shark | 4,000 |
| | (1526 g) | (1000 g) | | |
| Example 91. | N2-methyl- | Chondroitin sulfate | Bovine | 50,000 |
| | niacinamide | (1000 g) | | |
| | (136 g) | | | |
| Example 92. | N2-ethyl- | Chondroitin sulfate | Bovine | 50,000 |
| | niacinamide | (1000 g) | | |
| | (150 g) | | | |

### Example 93 to 100

### Weight ratio glucosaminoglycan/pyridine carboxy derivative 1: 4 (g/g).

| | | | | Average | Molar |
|---|---|---|---|---|---|
| | | | | molecular | ratio |
| | | | | weight | glucos- |
| | | | Origin | of the | amino- |
| | Pyridine | Glucos- | of the | glucos- | glycan/ |
| | carboxy | amino- | glucos- | amino- | pyridine |
| | derivative | glycan | amino- | glycan | carboxy |
| | (quantity) | (quantity) | glycan | (g/mol) | derivative |
| Example | Niacinamide | Chondroitin | Bovine | 4,000 | 1:131,0 |
| 93. | (4000 g) | sulfate | | | |
| | | (1000 g) | | | |
| Example | N2-ethyl- | Dermatan | Porcine | 4,000 | 1:106,7 |
| 94. | niacinamide | sulfate | | | |
| | (4000 g) | (1000 g) | | | |
| Example | N2-methyl- | Chondroitin | Shark | 4,000 | 1:117,6 |
| 95. | niacinamide | sulfate | | | |
| | (4000 g) | (1000 g) | | | |
| Example | Niacinamide | Heparin | Porcine | 6,000 | 1:196,6 |
| 96. | (4000 g) | (1000 g) | | | |
| Example | Niacinamide | Heparan | Bovine | 7,500 | 1:251,4 |
| 97. | (4000 g) | sulfate | | | |
| | | (1000 g) | | | |
| Example | Niacinamide | Chondroitin | Bovine | 50,000 | 1:1638,0 |
| 98. | (4000 g) | sulfate | | | |
| | | (1000 g) | | | |
| Example | N2-ethyl- | Keratan | Porcine | 30,000 | 1:800,8 |
| 99. | niacinamide | sulfate | | | |
| | (4000 g) | (1000 g) | | | |
| Example | N2-ethyl- | Chondroitin | Bovine | 17,000 | 1:453,5 |
| 100. | niacinamide | sulfate | | | |
| | (4000 g) | (1000 g) | | | |

### Example 101 to 108

### Weight ratio glucosaminoglycan/pyridine carboxy derivative 1:10 (g/g).

| | | | | Average | Molar |
|---|---|---|---|---|---|
| | | | | molecular | ratio |
| | | | | weight | glucos- |
| | | | Origin | of the | amino- |
| | Pyridine | Glucos- | of the | glucos- | glycan/ |
| | carboxy | amino- | glucos- | amino- | pyridine |
| | derivative | glycan | amino- | glycan | carboxy |
| | (quantity) | (quantity) | glycan | (g/mol) | derivative |
| Example | Niacinamide | Chondroitin | Bovine | 4,000 | 1:327,6 |
| 101. | (10,000 g) | sulfate | | | |
| | | (1000 g) | | | |
| Example | N2-ethyl- | Dermatan | Porcine | 4,000 | 1:266,7 |
| 102. | niacinamide | sulfate | | | |
| | (10,000 g) | (1000 g) | | | |
| Example | N2-methyl- | Chondroitin | Shark | 4,000 | 1:294,1 |
| 103. | niacinamide | sulfate | | | |
| | (10,000 g) | (1000 g) | | | |
| Example | Niacinamide | Heparin | Porcine | 6,000 | 1:511,9 |
| 104. | (10,000 g) | (1000 g) | | | |
| Example | Niacinamide | Heparan | Bovine | 7,500 | 1:615,8 |
| 105. | (10,000 g) | sulfate | | | |
| | | (1000 g) | | | |
| Example | Niacinamide | Chondroitin | Bovine | 50,000 | 1:4095,0 |
| 106. | (10,000 g) | sulfate | | | |
| | | (1000 g) | | | |
| Example | N2-ethyl- | Keratan | Porcine | 30,000 | 1:2002,0 |
| 107. | niacinamide | sulfate | | | |
| | (10,000 g) | (1000 g) | | | |
| Example | N2-ethyl- | Chondroitin | Bovine | 17,000 | 1:1133,8 |
| 108. | niacinamide | sulfate | | | |
| | (10,000 g) | (1000 g) | | | |

### General method example 109-133

The glucosaminoglycans are hygroscopic and are dried before use. A quantity of the glucosaminoglycan and the pyridine carboxy derivative are transferred to a hard gelatine capsule.

### Example 109 to 111

### Capsule 500 mg, molar ratio glucosaminoglycan/pyridine carboxy derivative 1:1000 (mol/mol).

| | | | | Average |
|---|---|---|---|---|
| | | | Origin | molecular |
| | Pyridine | | of the | weight of the |
| | carboxy | Glucosamino- | glucos- | glucos- |
| | derivative | glycan | amino- | aminoglycan |
| | (quantity) | (quantity/g) | glycan | (g/mol) |
| Example | Niacinamide | Chondroitin sulfate | Bovine | 17,000 |
| 109. | (439 mg) | (61 mg) | | |
| Example | Niacinamide | Chondroitin sulfate | Porcine | 30,000 |
| 110. | (401 mg) | (99 mg) | | |
| Example | N2-ethyl- | Chondroitin sulfate | Bovine | 17,000 |
| 111. | niacinamide | (99 mg) | | |
| | (449 mg) | | | |

### Example 112 to 114

### Capsule 250 mg, molar ratio glucosaminoglycan/pryridine carboxy derivative 1:500 (mol/mol).

| | | | | Average |
|---|---|---|---|---|
| | | | Origin | molecular |
| | Pyridine | | of the | weight of the |
| | carboxy | Glucosamino- | glucos- | glucos- |
| | derivative | glycan | amino- | aminoglycan |
| | (quantity) | (quantity) | glycan | (g/mol) |
| Example | Niacinamide | Chondroitin sulfate | Bovine | 17,000 |
| 112. | (196 mg) | (54 mg) | | |
| Example | Niacinamide | Chondroitin sulfate | Porcine | 30,000 |
| 113. | (168 mg) | (82 mg) | | |
| Example | N2-ethyl- | Chondroitin sulfate | Bovine | 17,000 |
| 114. | niacinamide | (46 mg) | | |
| | (204 mg) | | | |

### Example 115

### Capsule 500 mg, molar ratio glucosaminoglycan/pyridine carboxy derivative 1:500 (mol/mol).

| | | | | Average |
|---|---|---|---|---|
| | | | Origin | molecular |
| | Pyridine | | of the | weight of the |
| | carboxy | Glucosamino- | glucos- | glucos- |
| | derivative | glycan | amino- | aminoglycan |
| | (quantity) | (quantity) | glycan | (g/mol) |
| Example | Niacinamide | Heparan sulfate | Bovine | 7,500 |
| 115. | (445 mg) | (55 mg) | | |

### Example 116 to 118

### Capsule 250 mg, molar ratio glucosaminoglycan/pyridine carboxy derivative 1:200 (mol/mol).

| | | | Origin | Average |
|---|---|---|---|---|
| | Pyridine | | of the | molecular |
| | carboxy | Glucosamino- | glucos- | weight of the |
| | derivative | glycan | amino- | glucosamino- |
| | (quantity) | (quantity) | glycan | glycan (g/mol) |
| Example | Niacinamide | Chondroitin sulfate | Bovine | 17,000 |
| 116. | (147 mg) | (103 mg) | | |
| Example | Niacinamide | Chondroitin sulfate | Porcine | 30,000 |
| 117. | (112 mg) | (138 mg) | | |
| Example | N2-ethyl- | Chondroitin sulfate | Bovine | 17,000 |
| 118 | niacinamide | (91 mg) | | |
| | (159 mg) | | | |

### Example 119 to 122

### Capsule 500 mg, molar ratio glucosaminoglycan/pyridine carboxy derivative 1:200 (mol/mol).

| | | | Origin | Average |
|---|---|---|---|---|
| | Pyridine | | of the | molecular |
| | carboxy | Glucosamino- | glucos- | weight of the |
| | derivative | glycan | amino- | glucosamino- |
| | (quantity) | (quantity) | glycan | glycan (g/mol) |
| Example | Niacinamide | Chondroitin sulfate | Bovine | 17,000 |
| 119. | (295 mg) | (205 mg) | | |
| Example | Niacinamide | Chondroitin sulfate | Porcine | 30,000 |
| 120. | (224 mg) | (276 mg) | | |
| Example | Niacinamide | Heparin | Porcine | 6,000 |
| 121. | (401 mg) | (99 mg) | | |
| Example | N2-ethyl- | Chondroitin sulfate | Bovine | 17,000 |
| 122. | niacinamide | (181 mg) | | |
| | (319 mg) | | | |

### Example 123 to 125

### Capsule 250 mg, molar ratio glucosaminoglycan/pyridine carboxy derivative 1:100 (mol/mol).

| | | | Origin | Average |
|---|---|---|---|---|
| | Pyridine | | of the | molecular |
| | carboxy | Glucosamino- | glucos- | weight of the |
| | derivative | glycan | amino- | glucosamino- |
| | (quantity) | (quantity) | glycan | glycan (g/mol) |
| Example | Niacinamide | Heparin (82 mg) | Porcine | 6,000 |
| 123. | (168 mg) | | | |
| Example | Niacinamide | Heparan sulfate | Porcine | 7,500 |
| 124. | (155 mg) | (95 mg) | | |
| Example | N2-ethyl- | Chondroitin sulfate | Bovine | 17,000 |
| 125. | niacinamide | (91 mg) | | |
| | (159 mg) | | | |

### Example 126 to 133

### Capsule 250 mg, weight ratio glucosaminoglycan/pyridine carboxy derivative 1: 4 (g/g).

| | | | | Average | Molar |
|---|---|---|---|---|---|
| | | | | molecular | ratio |
| | | | | weight | glucos- |
| | | | Origin | of the | amino- |
| | Pyridine | Glucos- | of the | glucos- | glycan/ |
| | carboxy | amino- | glucos- | amino- | pyridine |
| | derivative | glycan | amino- | glycan | carboxy |
| | (quantity) | (quantity) | glycan | (g/mol) | derivative |
| Example | Niacinamide | Chondroitin | Bovine | 4,000 | 1:131,0 |
| 126. | (200 mg) | sulfate | | | |
| | | (50 mg) | | | |
| Example | N2-ethyl- | Dermatan | Porcine | 4,000 | 1:106,7 |
| 127. | niacin-amide | sulfate | | | |
| | (200 mg) | (50 mg) | | | |
| Example | N2-methyl- | Chondroitin | Shark | 4,000 | 1:117,6 |
| 128. | niacin-amide | sulfate | | | |
| | (200 mg) | (50 mg) | | | |
| Example | Niacinamide | Heparin | Porcine | 6,000 | 1:196,6 |
| 129. | (200 mg) | (50 mg) | | | |
| Example | Niacinamide | Heparan | Bovine | 7,500 | 1:251,4 |
| 130. | (200 mg) | sulfate | | | |
| | | (50 mg) | | | |
| Example | Niacinamide | Chondroitin | Bovine | 50,000 | 1:1638,0 |
| 131. | (200 mg) | sulfate | | | |
| | | (50 mg) | | | |
| Example | N2-ethyl- | Keratan | Porcine | 30,000 | 1:800,8 |
| 132. | niacin-amide | sulfate | | | |
| | (200 mg) | (50 mg) | | | |
| Example | N2-ethyl- | Chondroitin | Bovine | 17,000 | 1:453,5 |
| 133. | niacin-amide | sulfate | | | |
| | (200 mg) | (50 mg) | | | |

### Example 134

Study object

The immunomodulating and anti-inflammatory effects of complexes or compositions of the invention are tested in vitro. The model used is TNF-α secretion in human peripheral blood mononuclear leukocytes. Dexamethasone is employed as positive control.

Test Compounds

Any of the complexes or compositions according to examples 1 to 108 are tested.

Cellular TNF-α assay

The study is performed employing a modification of the methods of Page et al (Int. J. Oncology 3: 473-476, 1993) and Welker et al (Int. Arch. Of Allergy and Immunology, 109: 110-115, 1996). The test compound is dissolved in water or dimethylsulfoxide for the cellular assay. The test compounds are tested in duplicate at the following concentrations: 0.8 μg/ml, 4.0 μg/ml, 20.0 μg/ml, 100.0 μg/ml and 500.0 μg/ml.

The active compounds or DMSO 0.4% (control) are incubated with lipopolysaccharide-stimulated (25 ng/ml) human peripheral blood mononuclear leukocytes in growth medium RPMI-1640, pH 7.4 for 16 hours at 37° C. The TNF-α cytokine levels in the conditioned medium are quantitated using a sandwich ELISA kit.

Findings and interpretation

The complexes or compositions of the invention dose-dependently inhibit the secretion of TNF-α. Similar tests may be employed where other pro-inflammatory cytokines are measures, e.g. IL-1 β, IL-6 and IL-8. Similar tests may also be employed where pro-allergic cytokines are measures, e.g. IL-4, IL-5 or IL-13.

### EXAMPLE135

Study object

The anti-inflammatory effects of complexes or compositions of the invention are tested in vivo. The model employed is arthritis in the mouse induced by collagen monoclonal antibody (mAb) and lipopolysaccharide. Cyclophosphamide is used as positive control.

Test Compounds

Any of the complexes or compositions according to examples 1 to 108 are tested.

Dosing pattern

The test compound is dissolved in water. The test compound is administered orally once daily for 3 consecutive days at 100, 300 and 1000 mg/kg. The positive control (cyclophosphamide) is administered at 10 mg/kg. Dosing volume is 5 mi/kg.

Animals

In this study, male BALB/c mice weighing 20 ±1 grams are used. Space allocation for 5 mice is 45×23×15 cm. The animals are housed in APEC® (Allentown Gaging, Allentown, N.J. 08501, U. S. A.) cages and maintained in a hygienic environment under controlled temperature (22° C. -24° C.) and humidity (60% -80%) with 12-hours light/dark cycles for at least one week prior to being used. Free access to standard lab chow for mice and tap water is granted. All aspects of this work including housing, experimentation and disposal of animals is performed in general according to the International Guiding Principles for Biomedical Research Involving Animals (CIOMS Publication No. ISBN 92 90360194, 1985).

Chemicals

The chemicals employed in the present study are standard chemicals e.g. Cyclo-phosphamide (Sigma, U. S. A.), Lipopolysaccharide (Sigma, U. S. A.), and Arthrogen-CIA™ Monoclonal Antibodies D8, F10, DI-2G and A2 (Chondrex, U. S. A.).

Equipment

Equipment employed is a Plethysmometer (Ugo Basile, U. S. A.),

Method

The study is performed according to the method of Terato et al (Autoimmunity, 22: 137-147, 1995).

Groups of 5 BALB/c mice, 6-8 weeks of age, are used for the induction of arthritis by monoclonal antibodies (mAbs) and lipopolysaccharide (LPS). The animals are administered intravenously of a combination of 4 different mAbs (D8, F10, DI-2G and A2) in a total of 4 mg/mouse at day 0. This is followed by intravenous 25 μg of LPS 72 hours later (day 3). From day 3, test substances are administered orally once daily for 3 consecutive days. A plethysmometer (Ugo Basile Cat # 7150) with water cell (12 mm diameter) is used for the measurement of increase in both hind paw volumes at day 7, 10, 14 and 17. The percent of inhibition of increased paw volume is calculated as follows:

Inhibition (%): [1−(Tn−To)/(Cn−Co)]×100

Where:

Co (Cn): Volume of day 0 (day n) in vehicle control (both hind paws summed) To (Tn): Volume of day 0 (day n) in test compound-treated group (both hind paws summed)

Findings and interpretation

THE COMPLEXES OR COMPOSITIONS OF THE INVENTION DOSE-DEPENDENTLY INHIBIT PAW SWELLING.

1. A chemical complex comprising:
i) a substituted or unsubstituted pyridine carboxy derivative according to formula 1 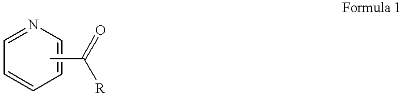
wherein R is selected from the group consisting of OH; OR′; NH₂; NHR′; NR′R″, O⁻Y⁺, and halogen, wherein R′ and R″ are independently selected from substituted or unsubstituted C₁-C₂₀ alkyl; and Y is a base addition salt of the free carboxylate; and
ii) a glucosaminoglycan or a fragment or derivative thereof.

2. A chemical complex according to claim 1, wherein the glucosaminoglycan is selected from the group consisting of chondroitin sulfates, dermatan sulfates, keratan sulfates, hyaluronic acids and fragments or derivatives thereof.

3. A chemical complex according to claim 1, wherein the glucosaminoglycan consists of a repeat region, linkage region and a chain cap wherein the repeat region comprises at least one disaccharide unit in which one or both of the sugar monomers of said disaccharide unit is either galactosamine or glucosamine, such as N-acetylgalactosamine or N-acetylglucosamine;
the linkage region is present at least once, is linked to the repeat region, may be a di-, oligo or poly-saccharide or a di-, oligo or poly-saccharide chain with a terminal amino acid; and is suitable for linking to a protein; and
the cap region is a di-, oligo or poly-saccharide present at least once and is linked to the repeat region.

4. A chemical complex according to claim 3, wherein the repeat unit comprises any combination of disaccharides according to Formula 2 to 8.

5. A chemical complex according to any one of claims 1 to 4, wherein the glucosaminoglycan has a molecular weight of 5000 to 1000000 Da.

6. A chemical complex according to any one of claims 1 to 4, wherein the glucosaminoglycan has a molecular weight below 5000 Da.

7. A chemical complex according to claim 3, wherein the linkage unit is selected from the group consisting of -4(GlcAβ(1-3)Galβ(1-3)Galβ(1-4)Xylβ(1-0)-Ser and -4(GlcAβ(1-3)Galβ(1-3)Galβ(1-4)Xylβ(1-0)- where in C-4 and C-6 in Galβ independently may be SO₃ or OH.

8. A chemical complex according to any one of claims 1 to 4, wherein the pyridine carboxy derivative is niacinamide.

9. A chemical complex according to any one of claims 1 to 4, wherein the molar ratio or weight ratio between the pyridine carboxy derivative of formula I and the glucosaminoglycan is in the range of 1:100000 to 100000:1.

10. A chemical complex according to any one of claims 1 to 4, further comprising a pharmaceutically acceptable carrier.

11. A pharmaceutical composition, a dietary supplement or a cosmetic comprising a chemical complex as defined in any one of claims 1 to 4.

12. The use of a chemical complex as defined in claims 1, or a composition comprising said complex for:
(a) the preparation of a medicament, a dietary supplement or a cosmetic for immunomodulation in a mammal;
(b) the preparation of a medicament, a dietary supplement or a cosmetic for the suppression of hypersensitivity and/or inflammatory reaction in a mammal;
(c) the preparation of a medicament, a dietary supplement or a cosmetic for the treatment or prevention of autoimmune disease and/or chronic inflammatory disease in a mammal;
(d) the preparation of a medicament, a dietary supplement or a cosmetic for the treatment or prevention of psoriasis, atopic dermatitis, Crohn's disease, ulcerative colitis, rheumatoid arthritis, gout and/or osteoarthritis in a mammal;
(e) the preparation of a medicament, a dietary supplement or a cosmetic for the alleviation of pain in a mammal; or
(f) the preparation of a medicament, a dietary supplement or a cosmetic for chondroprotection in a mammal.

13. The use according to claim 12, wherein the mammal is a human.

14. A method for the treatment or prevention of hypersensitivity or inflammation in a mammal, which comprises administering a chemical complex as defined in claim 1, or a composition comprising said complex to said mammal.

15. A method for the treatment or prevention of an autoimmune disorder and/or a chronic inflammatory disorder in a mammal, which comprises administering a chemical complex as defined in claim 1, or a composition comprising said complex to said mammal.

16. A method for the treatment or prevention of psoriasis, atopic eczema, Crohn's disease, ulcerative colitis, rheumatoid arthritis, gout and/or osteoarthritis in a mammal, which comprises administering a chemical complex as defined in claim 1, or a composition comprising said complex to said mammal.

17. A method for the treatment or prevention of pain in a mammal, which comprises administering a chemical complex as defined in claim 1, or a composition comprising said complex to said mammal.

18. A method according to any one of claims 14-17, wherein the mammal is a human.
